(19) United States
(12) Patent Application Publication
(10) Pub. No.: US 2003/0176710 A1
(43) Pub. Date: Sep. 18, 2003

(54) C1-C6-epothilone fragments and process for the production of C1-C6-fragments of epothilones and derivatives thereof

(75) Inventors: Ulrich Klar, Berlin (DE); Markus Berger, Berlin (DE); Bernd Buchmann, Hohen Neuendorf (DE); Wolfgang Schwede, Berlin (DE); Werner Skuballa, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Schering AG, Berlin (DE)

(21) Appl. No.: 10/326,263

(22) Filed: Dec. 23, 2002

(30) Foreign Application Priority Data
Dec. 21, 2001 (DE) 10164592.9

(51) Int. Cl. C07D 417/02; C07D 277/22

(52) U.S. Cl. 548/181; 548/203

(57) ABSTRACT

This invention describes C₁-C₆-epothilone fragments and an efficient process for the production of C₁-C₆-fragments of epothilones and derivatives thereof.

Höfle et al. describe the cytotoxic action of the natural substances epothilone A (R=hydrogen) and epothilone B (R=methyl)

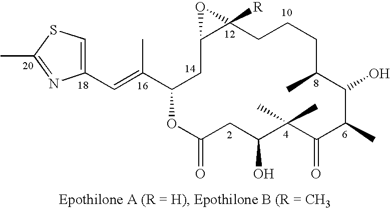

in, e.g., Angew. Chem. [Applied Chem.] 1996, 108, 1671-1673. Because of the in-vitro selectivity for breast cell lines and intestinal cell lines and their significantly higher activity against P-glycoprotein-forming multiresistant tumor lines in comparison to taxol as well as their physical properties that are superior to those of taxol, e.g., a water solubility that is higher by a factor of 30, this novel structural class is especially advantageous for the development of a pharmaceutical agent for therapy of malignant tumors.

The object of this invention consists in making available new C1-C6-epothilone components in large quantities that can be used for the synthesis of a wide variety of epothilones and derivates thereof, as they are described in, for example, WO 99/07692, WO 00/49020, WO 00/01333 or DE 199210861.

Slightly altering, for example, the process that is described in WO 99/07692, it has been shown, surprisingly enough, that by using a heretofore unmentioned protective group combination, a significant improvement of the synthesis both under economical and ecological aspects is possible.

This invention describes the new C₁-C₆-epothilone fragments of general formula I,

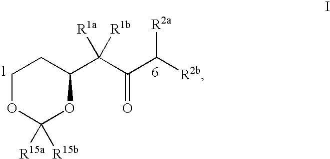

in which

R^{1a}, R^{1b} are the same or different and mean hydrogen, C₁-C₁₀-alkyl, aryl, C₇-C₂₀-aralkyl, or together mean a —(CH₂)ₘ group with m=2, 3, 4 or 5,

R^{2a}, R^{2b} are the same or different and mean hydrogen, C₁-C₁₀-alkyl, C₂-C₁₀-alkenyl, C₂-C₁₀-alkinyl, aryl, C₇-C₂₀-aralkyl or together mean a —(CH₂)ₙ group with n=2, 3, 4 or 5,

R^{15a}, R^{15b} are the same or different and mean hydrogen, C₁-C₁₀-alkyl, aryl, C₇-C₂₀-aralkyl, or together a —(CH₂)_{q} group,

q means 3 to 6,

including all stereoisomers as well as mixtures thereof.

As alkyl groups R^{1a}, R^{1b}, R^{2a}, R^{2b}, R^{15a}, and R^{15b}, straight-chain or branched-chain alkyl groups with 1-10 carbon atoms can be considered, such as, for example, methyl, ethyl, propyl, isopropyl, butyl, isobutyl, tert.-butyl, pentyl, isopentyl, neopentyl, heptyl, hexyl, and decyl.

Alkyl groups R^{1a}, R^{1b}, R^{2a}, R^{2b}, R^{15a} and R^{15b} can be perfluorinated or substituted by 1-5 halogen atoms, hydroxy groups, C₁-C₄-alkoxy groups, or C₆-C₁₂-aryl groups (which can be substituted by 1-3 halogen atoms).

As aryl radicals R^{1a}, R^{1b}, R^{2a}, R^{2b}, R^{15a} and R^{15b}, substituted and unsubstituted carbocyclic or heterocyclic radicals with one or more heteroatoms, such as, e.g., phenyl, naphthyl, furyl, thienyl, pyridyl, pyrazolyl, pyrimidinyl, oxazolyl, pyridazinyl, pyrazinyl, quinolyl, and thiazolyl, which can be substituted in one or more places by halogen, OH, O-alkyl, CO₂H, CO₂-alkyl, —NH₂, —NO₂, —N₃, —CN, C₁-C₂₀-alkyl, C₁-C₂₀-acyl, and C₁-C₂₀-acyloxy groups, are suitable.

The aralkyl groups in R^{1a}, R^{1b}, R^{2a}, R^{2b}, R^{15a} and R^{15b} can contain in the ring up to 14 C atoms, preferably 6 to 10 C atoms, and in the alkyl chain 1 to 8 atoms, preferably 1 to 4 atoms. As aralkyl radicals, for example, benzyl, phenylethyl, naphthylmethyl, naphthylethyl, furylmethyl, thienylethyl, and pyridylpropyl are considered. The rings can be substituted in one or more places by halogen, OH, O-alkyl, CO₂H, CO₂-alkyl, —NO₂, —N₃, —CN, C₁-C₂₀-alkyl, C₁-C₂₀-acyl, and C₁-C₂₀-acyloxy groups.

The acyl groups in R^{1a}, R^{1b}, R^{2a}, R^{2b}, R^{15a} and R^{15b} can contain 1 to 10 carbon atoms, whereby formyl, acetyl, propionyl, isopropionyl and pivalyl groups are preferred.

As alkenyl groups R^{2a} and R^{2b}, straight-chain or branched-chain alkyl groups with 1-10 carbon atoms can be considered, in which at least one C—C bond is replaced by a C═C bond, such as, for example, propenyl, butenyl, isobutenyl, pentenyl, isopentenyl, neopentenyl, heptenyl, heptadienyl, decenyl, or decatrienyl.

As alkinyl groups R^{2a} and R^{2b}, straight-chain or branched-chain alkyl groups with 1-10 carbon atoms can be considered, in which at least one C—C bond is replaced by a C═C bond, such as, for example, propinyl, butinyl, pentinyl, isopentinyl, heptinyl, heptadiinyl, decinyl, and decatriinyl.

Preferred are those compounds I in which

R^{1a}, R^{1b} are the same and mean C₁-C₆-alkyl, or together mean a —(CH₂)ₘ group with m=2, 3 or4,

R^{2a}, R^{2b} are different and mean hydrogen, C₁-C₆-alkyl, C₂-C₁₀-alkenyl, C₂-C₁₀-alkinyl or C₇-C₂₀-aralkyl,

R^{15a}, R^{15b} are the same or different and mean hydrogen, C₁-C₅-alkyl, aryl, or C₇-C₂₀-aralkyl, or together mean a —(CH₂)_{q} group,

q means 3 to 6.

Especially preferred are those compounds I in which

R^{1a}, R^{1b} are the same and mean methyl, ethyl, aryl, or together mean a —(CH₂)ₘ group with m=2 or 3,

R^{2a} means hydrogen,

R^{2b} means C₁-C₅-alkyl, C₂-C₆-alkenyl or C₂-C₆-alkinyl,

R^{15a}, R^{15b} are the same and mean C₁-C₃-alkyl, or together mean a —(CH₂)_{q} group, or

R^{15a} means hydrogen, and

R^{15b} means aryl,

q means 4 or 5.

Partial fragments A, in which R^{1a}′=R^{1b}′=methyl, can be efficiently produced from inexpensive pantolactone with an optical purity of >98%.

The process according to the invention is described in Diagram 1 below in the example of D-(−)-pantolactone. Enantiomer compounds ent-A-II to ent-A-XIV that correspond to A-II to A-XIV are obtained from L-(+)-pantolactone, and the corresponding racemic compounds rac-A-II to rac-A-XIV are obtained from racemic DL-pantolactone:

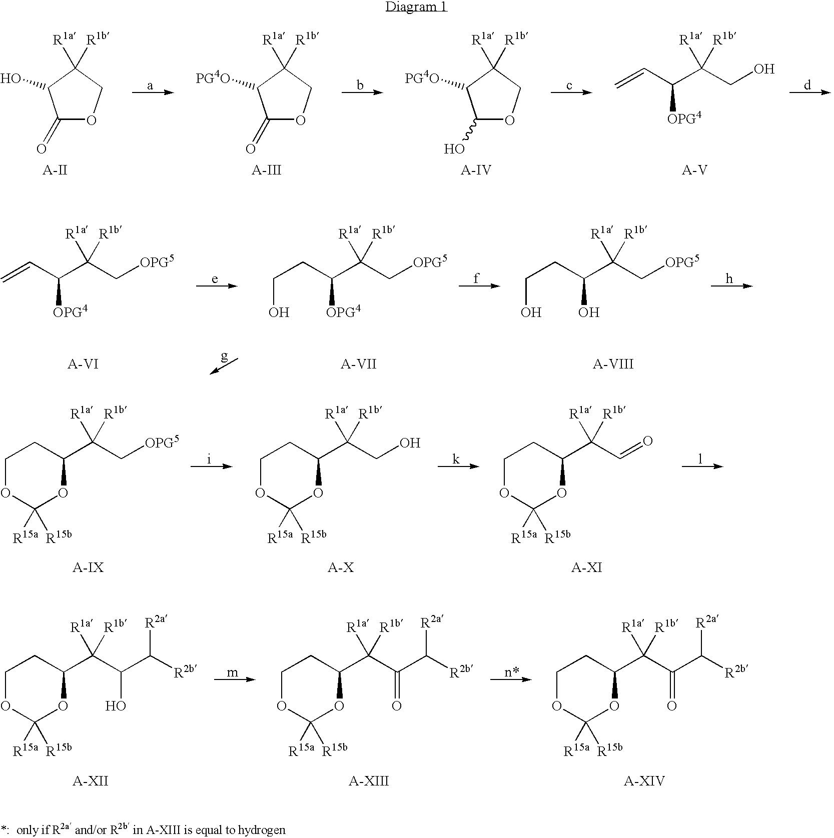

Step a (A-II [symbol] A-III):

The free hydroxy group of pantolactone (A-II) is protected according to the methods that are known to one skilled in the art. As protective group PG⁴, the protective groups that are known to one skilled in the art, such as, e.g., methoxymethyl, methoxyethyl, ethoxyethyl, tetrahydropyranyl, tetrahydrofuranyl, trimethylsilyl, triethylsilyl, tert.-butyldimethylsilyl, tert.-butyldiphenylsilyl, tribenzylsilyl, triisopropylsilyl, benzyl, para-nitrobenzyl, para-methoxybenzyl, formyl, acetyl, propionyl, isopropionyl, pivalyl, butyryl or benzoyl radicals, are suitable.

A survey is found in, e.g., “Protective Groups in Organic Synthesis” Theodora W. Green, John Wiley and Sons).

Preferred are those protective groups that can be cleaved under acidic reaction conditions, such as, e.g., methoxymethyl, tetrahydropyranyl, tetrahydrofuranyl, and trimethylsilyl radicals.

Especially preferred is the tetrahydropyranyl radical.

Step b (A-III [symbol] A-IV):

The protected lactone A-III is reduced to lactol A-IV. As a reducing agent, aluminum hydrides that are modified in their reactivity, such as, e.g., diisobutylaluminum hydride, are suitable. The reaction is carried out in an inert solvent such as, e.g., toluene, preferably at low temperatures.

Step c (A-IV [symbol] A-V):

Lactol A-IV is opened up to form hydroxyolefin A-V while expanding by one C atom. For this purpose, the methods that are known to one skilled in the art, such as, e.g., olefination according to Tebbe, the Wittig reaction or Wittig/Horner reaction, and the addition of an organometallic compound while being cleaved with water, are suitable. Preferred is the Wittig reaction with use of methyltriarylphosphonium halides, such as, e.g., methyltriphenylphosphonium bromide, with strong bases such as, e.g., n-butyllithium, potassium-tert-butanolate, sodium ethanolate, or sodium hexamethyl disilazane; n-butyllithium is preferred as a base.

Step d (A-V [symbol] A-VI):

The free hydroxy group in A-V is protected according to the methods that are known to one skilled in the art. As protective group PG⁵, the protective groups that are known to one skilled in the art, as they were already mentioned above for PG⁴ in Step a (A-II [symbol] A-III), are suitable.

Preferred are those protective groups that can be cleaved hydrogenolytically with use of the catalysts that are familiar to one skilled in the art, such as, e.g., benzyl, nitrobenzyl, methoxybenzyl, or benzyl radicals that are substituted in some other way.

The benzyl radical is especially preferred.

Step e (A-VI [symbol] A-VIII):

Water is added to the double bond in A-VI in an anti-Markovnikov orientation. For this purpose, the processes that are known to one skilled in the art, such as, e.g., the reaction with boranes, their subsequent oxidation to the corresponding boric acid esters and their saponification, are suitable. As boranes, e.g., the borane-tetrahydrofuran complex, the borane-dimethyl sulfide complex, and 9-borabicyclo[3.3.1]nonane in an inert solvent, such as, for example, tetrahydrofuran or diethyl ether, are preferred. As an oxidizing agent, preferably hydrogen peroxide is used, and for saponification of boresters, preferably alkali hydroxides, such as, e.g., sodium hydroxide, are used.

Step f (A-VI [symbol] A-VII):

Protective group PG⁴ that is introduced under step a) is now cleaved according to the process that is known to one skilled in the art. If this is a protective group that can be cleaved acidically, then cleavage can be accomplished with dilute mineral acids in aqueous-alcoholic solutions and with the aid of catalytic quantities of acids, such as, e.g., para-toluenesulfonic acid, para-toluenesulfonic acid-pyridinium salt, camphorsulfonic acid in alcoholic solutions, preferably in ethanol or isopropanol.

Step g (A-VII [symbol] A-IX):

A common protection of the two alcohol functions of the mono-protected 1.3-diol in A-VII is possible under acid catalysis by direct ketalization with a carbonyl compound of general formula R^{15a}—CO—R^{15b}, or by reketalization with a ketal of general formulas R^{15a}—C(OC₂H₅)₂—R^{15b}, R^{15a}—C(OC₂H₄)₂—R^{15b}, and R^{15a}—C(OCH₂C(CH₃)₂CH₂O)—R^{15b} in which in each case R^{15a} and R^{15b} have the above-indicated meanings. As acids, the acids that are already mentioned under step f) are suitable; the use of para-toluenesulfonic acid optionally with the addition of copper(II) salts or cobalt(II) salts, such as, e.g., copper (II) sulfate, is preferred.

Step h (A-VIII [symbol] A-IX):

Protection of both alcohol functions of the 1.3-diol in A-VIII is possible under acid catalysis by direct ketalization with a carbonyl compound of general formula R^{15a}—CO—R^{15b}, or by reketalization with a ketal of the general formulas R^{15a}—C(OC₂H₅)₂—R^{15b}, R^{15a}—C(OC₂H₄)₂—R^{15b}, and R^{15a}—C(OCH₂C(CH₃)₂CH₂O)—R^{15b} in which in each case R^{15a} and R^{15b} have the above-indicated meanings. Reketalization preferably with 2,2-dimethoxypropane is preferred. As acids, the acids that are already mentioned under step f) are suitable; the use of camphorsulfonic acid is preferred.

Step i (A-IX [symbol] A-X):

Protective group PG⁵ that is introduced under step d) is now cleaved according to the processes that are known to one skilled in the art. If this is an optionally substituted benzyl ether, the latter is cleaved with hydrogen in the presence of a suitable catalyst.

Hydrogen pressures of 1 to 100 atm, especially preferably 1-10 atm, are preferred for the cleavage.

As catalysts, the catalysts that are based on palladium, rhodium, nickel or platinum and that are known to one skilled in the art are suitable.

Palladium on carbon or platinum in the form of PtO₂ is preferred.

Palladium on carbon is especially preferred.

Step k (A-X [symbol] A-XI):

The oxidation of the primary alcohol in A-X to aldehyde is carried out according to the methods that are known to one skilled in the art. For example, the oxidation with pyridinium chlorochromate, pyridinium dichromate, chromium trioxide-pyridine complex, the oxidation according to Swem or related methods, e.g., with use of oxalyl chloride in dimethyl sulfoxide, the use of Dess-Martin-periodinane, the use of nitrogen oxides, such as, e.g., N-methyl-morpholino-N-oxide in the presence of suitable catalysts, such as, e.g., tetrapropylammonium perruthenate in inert solvents, can be mentioned. The oxidation according to Swem or the use of SO₃-pyridine as well as with N-methyl-morpholino-N-oxide with use of tetrapropylammonium perruthenate is preferred.

Step l (A-XI [symbol] A-XII):

The reaction of aldehyde A-XI to form alcohols of formula A-XII is carried out with organometallic compounds of general formula M—CHR^{2a}′R^{2b}′, in which M stands for an alkali metal, preferably lithium, or a divalent metal MX, in which X represents a halogen, and radicals R^{2a′} and R^{2b′} in each case have the above-mentioned meanings. As a divalent metal, magnesium and zinc is preferred; as a halogen, X is preferably chlorine, bromine or iodine.

Step m (A-XII [symbol] A-XIII):

The oxidation of the secondary alcohol in A-XII to ketone A-XIII is carried out according to the conditions that are mentioned under step k). The oxidation according to Swem or the use of SO₃-pyridine as well as with N-methyl-morpholino-N-oxide with use of tetrapropylammonium perruthenate is preferred.

Step n (A-XIII [symbol] A-XIV):

In the event that R^{2a}′ and/or R^{2b}′ in A-XIII is equal to hydrogen, the possibility exists of introducing for this purpose a second radical R^{2a}′ that has the above-mentioned meanings, excluding hydrogen. In this connection, with use of strong bases, such as, e.g., lithium diisopropylamide, the ketone in A-XIII is converted into the enolate and reacted with a compound of general formula X-R^{2a}′, in which X represents a halogen. The addition of a chelating agent, such as, for example, 1,3-dimethyltetrahydro-2(1H)-pyrimidinone is optionally recommended. As a halogen, X is preferably chlorine, bromine and iodine.

In contrast to the process that is described in, for example, WO 99/07692, significant improvements are achieved by the procedure that is described here:

The currently preferred protective group PG⁵, the cost-intensive t-butyl-diphenylsilyl ether, is replaced by a reasonably-priced, optionally substituted benzyl protective group.

By using an optionally substituted benzyl protective group for PG⁵, a solvent-intensive purification of stages A-VI, A-X and A-XI by chromatography can become completely unnecessary.

The hydroboration with the borane-THF complex is now possible in a better yield in the presence of the THP protective group for PG⁴ and an optionally substituted benzyl protective group for PG⁵.

The quantity of borane-THF complex for the AVI transformation after A-VII can be reduced from 3.0 to 0.6 molar equivalents. In the same way, the quantities of hydrogen peroxide and alkaline base can be reduced.

In this new process, A-VII can be converted directly into A-IX.

The methyl ketone A-XVIII (R^{2a′}=R^{2b′}=H) can be purified by crystallization; a difficult and costly chromatography step is no longer necessary.

A-XIV can be obtained by simple alkylation of ketone A-XIII with alkyl, alkenyl or alkinyl halides that are inexpensive or simple to produce.

As early as in the research laboratory, kilogram quantities of the component A-XIV can be produced according to this new process.

The C₁-C₆-fragments A-XIII that are mentioned below are preferred according to the invention:

(4S)-4-(2-Methyl-3-oxo-pent-2-yl)-2,2-dimethyl-[1,3]dioxane

(4S)-4-(2-Methyl-3-oxo-hex-2-yl)-2,2-dimethyl-[1,3]dioxane

(4S)-4-(2-Methyl-3-oxo-hept-2-yl)-2,2-dimethyl-[1,3]dioxane

(4S)-4-(2-Methyl-3-oxo-hept-2-yl)-2,2-dimethyl-[1,3]dioxane

(4S)-4-(2-Methyl-3-oxo-hex-5-en-2-yl)-2,2-dimethyl-[1,3]dioxane

(4S)-4-(2-Methyl-3-oxo-hept-6-en-2-yl)-2,2-dimethyl-[1,3]dioxane

(4S)-4-(2-Methyl-3-oxo-oct-6-en-2-yl)-2,2-dimethyl-[1,3]dioxane

(4S)-4-(2-Methyl-3-oxo-oct-7-en-2-yl)-2,2-dimethyl-[1,3]dioxane

(4S)-4-(2-Methyl-3-oxo-hex-5-in-2-yl)-2,2-dimethyl-[1,3]dioxane

(4S)-4-(2-Methyl-3-oxo-hept-6-in-2-yl)-2,2-dimethyl-[1,3]dioxane

(4S)-4-(2-Methyl-3-oxo-oct-6-in-2-yl)-2,2-dimethyl-[1,3]dioxane

(4S)-4-(2-Methyl-3-oxo-oct-7-in-2-yl)-2,2-dimethyl-[1,3]dioxane

(4S)-4-(2-Methyl-3-oxo-pent-2-yl)-2,2-(1,4-tetramethylene)-[1,3]dioxane

(4S)-4-(2-Methyl-3-oxo-hex-2-yl)-2,2-(1,4-tetramethylene)-[1,3]dioxane

(4S)-4-(2-Methyl-3-oxo-hept-2-yl)-2,2-(1,4-tetramethylene)-[1,3]dioxane

(4S)-4-(2-Methyl-3-oxo-hept-2-yl)-2,2-(1,4-tetramethylene)-[1,3]dioxane

(4S)-4-(2-Methyl-3-oxo-hex-5-en-2-yl)-2,2-(1,4-tetramethylene)-[1,3]dioxane

(4S)-4-(2-Methyl-3-oxo-hept-6-en-2-yl)-2,2-(1,4-tetramethylene)-[1,3]dioxane

(4S)-4-(2-Methyl-3-oxo-oct-6-en-2-yl)-2,2-(1,4-tetramethylene)-[1,3]dioxane

(4S)-4-(2-Methyl-3-oxo-oct-7-en-2-yl)-2,2-(1,4-tetramethylene)-[1,3]dioxane

(4S)-4-(2-Methyl-3-oxo-hex-5-in-2-yl)-2,2-(1,4-tetramethylene)-[1,3]dioxane

(4S)-4-(2-Methyl-3-oxo-hept-6-in-2-yl)-2,2-(1,4-tetramethylene)-[1,3]dioxane

(4S)-4-(2-Methyl-3-oxo-oct-6-in-2-yl)-2,2-(1,4-tetramethylene)-[1,3]dioxane

(4S)-4-(2-Methyl-3-oxo-oct-7-in-2-yl)-2,2-(1,4-tetramethylene)-[1,3]dioxane

(4S)-4-(2-Methyl-3-oxo-pent-2-yl)-2,2-(1,5-pentamethylene)-[1,3]dioxane

(4S)-4-(2-Methyl-3-oxo-hex-2-yl)-2,2-(1,5-pentamethylene)-[1,3]dioxane

(4S)-4-(2-Methyl-3-oxo-hept-2-yl)-2,2-(1,5-pentarnethylene)-[1,3]dioxane

(4S)-4-(2-Methyl-3-oxo-hept-2-yl)-2,2-(1,5-pentamethylene)-[1,3]dioxane

(4S)-4-(2-Methyl-3-oxo-hex-5-en-2-yl)-2,2-(1,5-pentamethylene)-[1,3]dioxane

(4S)-4-(2-Methyl-3-oxo-hept-6-en-2-yl)-2,2-(1,5-pentamethylene)-[1,3]dioxane

(4S)-4-(2-Methyl-3-oxo-oct-6-en-2-yl)-2,2-(1,5-pentamethylene)-[1,3]dioxane

(4S)-4-(2-Methyl-3-oxo-oct-7-en-2-yl)-2,2-(1,5-pentamethylene)-[1,3]dioxane

(4S)-4-(2-Methyl-3-oxo-hex-5-in-2-yl)-2,2-(1,5-pentamethylene)-[1,3]dioxane

(4S)-4-(2-Methyl-3-oxo-hept-6-in-2-yl)-2,2-(1,5-pentamethylene)-[1,3]dioxane

(4S)-4-(2-Methyl-3-oxo-oct-6-in-2-yl)-2,2-(1,5-pentamethylene)-[1,3]dioxane

(4S)-4-(2-Methyl-3-oxo-oct-7-in-2-yl)-2,2-(1,5-pentamethylene)-[1,3]dioxane

(4S,2RS)-4-(2-Methyl-3-oxo-pent-2-yl)-2-phenyl-[1,3]dioxane

(4S,2RS)-4-(2-Methyl-3-oxo-hex-2-yl)-2-phenyl-[1,3]dioxane

(4S,2RS)-4-(2-Methyl-3-oxo-hept-2-yl)-2-phenyl-[1,3]dioxane

(4S,2RS)-4-(2-Methyl-3-oxo-hept-2-yl)-2-phenyl-[1,3]dioxane

(4S,2RS)-4-(2-Methyl-3-oxo-hex-5-en-2-yl)-2-phenyl-[1,3]dioxane

(4S,2RS)-4-(2-Methyl-3-oxo-hept-6-en-2-yl)-2-phenyl-[1,3]dioxane

(4S,2RS)-4-(2-Methyl-3-oxo-oct-6-en-2-yl)-2-phenyl-[1,3]dioxane

(4S,2RS)-4-(2-Methyl-3-oxo-oct-7-en-2-yl)-2-phenyl-[1,3]dioxane

(4S,2RS)-4-(2-Methyl-3-oxo-hex-5-in-2-yl)-2-phenyl-[1,3]dioxane

(4S,2RS)-4-(2-Methyl-3-oxo-hept-6-in-2-yl)-2-phenyl-[1,3]dioxane

(4S,2RS)-4-(2-Methyl-3-oxo-oct-6-in-2-yl)-2-phenyl-[1,3]dioxane

(4S,2RS)-4-(2-Methyl-3-oxo-oct-7-in-2-yl)-2-phenyl-[1,3]dioxane

(4S,2RS)-4-(2-Methyl-3-oxo-pent-2-yl)-2-(4-methoxy-phenyl)-[1,3]dioxane

(4S,2RS)-4-(2-Methyl-3-oxo-hex-2-yl)-2-(4-methoxy-phenyl)-[1,3]dioxane

(4S,2RS)-4-(2-Methyl-3-oxo-hept-2-yl)-2-(4-methoxy-phenyl)-[1,3]dioxane

(4S,2RS)-4-(2-Methyl-3-oxo-hept-2-yl)-2-(4-methoxy-phenyl)-[1,3]dioxane

(4S,2RS)-4-(2-Methyl-3-oxo-hex-5-en-2-yl)-2-(4-methoxy-phenyl)-[1,3]dioxane

(4S,2RS)-4-(2-Methyl-3-oxo-hept-6-en-2-yl)-2-(4-methoxy-phenyl)-[1,3]dioxane

(4S,2RS)-4-(2-Methyl-3-oxo-oct-6-en-2-yl)-2-(4-methoxy-phenyl)-[1,3]dioxane

(4S,2RS)-4-(2-Methyl-3-oxo-oct-7-en-2-yl)-2-(4-methoxy-phenyl)-[1,3]dioxane

(4S,2RS)-4-(2-Methyl-3-oxo-hex-5-in-2-yl)-2-(4-methoxy-phenyl)-[1,3]dioxane

(4S,2RS)-4-(2-Methyl-3-oxo-hept-6-in-2-yl)-2-(4-methoxy-phenyl)-[1,3]dioxane

(4S,2RS)-4-(2-Methyl-3-oxo-oct-6-in-2-yl)-2-(4-methoxy-phenyl)-[1,3]dioxane

(4S,2RS)-4-(2-Methyl-3-oxo-oct-7-in-2-yl)-2-(4-methoxy-phenyl)-[1,3]dioxane

(4S,2RS)-4-(2-Methyl-3-oxo-pent-2-yl)-2-(2-cyano-phenyl)-[1,3]dioxane

(4S,2RS)-4-(2-Methyl-3-oxo-hex-2-yl)-2-(2-cyano-phenyl)-[1,3]dioxane

(4S,2RS)-4-(2-Methyl-3-oxo-hept-2-yl)-2-(2-cyano-phenyl)-[1,3]dioxane

(4S,2RS)-4-(2-Methyl-3-oxo-hept-2-yl)-2-(2-cyano-phenyl)-[1,3]dioxane

(4S,2RS)-4-(2-Methyl-3-oxo-hex-5-en-2-yl)-2-(2-cyano-phenyl)-[1,3]dioxane

(4S,2RS)-4-(2-Methyl-3-oxo-hept-6-en-2-yl)-2-(2-cyano-phenyl)-[1,3]dioxane

(4S,2RS)-4-(2-Methyl-3-oxo-oct-6-en-2-yl)-2-(2-cyano-phenyl)-[1,3]dioxane

(4S,2RS)-4-(2-Methyl-3-oxo-oct-7-en-2-yl)-2-(2-cyano-phenyl)-[1,3]dioxane

(4S,2RS)-4-(2-Methyl-3-oxo-hex-5-in-2-yl)-2-(2-cyano-phenyl)-[1,3]dioxane

(4S,2RS)-4-(2-Methyl-3-oxo-hept-6-in-2-yl)-2-(2-cyano-phenyl)-[1,3]dioxane

(4S,2RS)-4-(2-Methyl-3-oxo-oct-6-in-2-yl)-2-(2-cyano-phenyl)-[1,3]dioxane

(4S,2RS)-4-(2-Methyl-3-oxo-oct-7-in-2-yl)-2-(2-cyano-phenyl)-[1,3]dioxane

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius, and all parts and percentages are by weight, unless otherwise indicated.

### EXAMPLE 1

(4S)-4-(2-Methyl-3-oxo-hept-6-en-2-yl)-2,2-dimethyl-[1,3]dioxane

### Example 1a

(3S)-1-Benzyloxy-2,2-dimethyl-3-(tetrahydropyran-2(RS)-yloxy)-pent-4-ene

The solution of (3S)-1-hydroxy-2,2-dimethyl-3-(tetrahydropyran-2(RS)-yloxy)-pent-4-ene (1475 g, 6883 mmol), which was produced analogously to the process that is described in WO 99/07692, in dioxane (2 l) is added to a suspension of KO-t-Bu (1600 g, 14258 mmol) in dioxane (11 l) over a period of 2 hours. After 2 hours, benzyl bromide (910 ml, 7651 mmol) is added over a period of 75 minutes. The mixture is stirred at 23° C. overnight, mixed with saturated ammonium chloride solution, water is added (5 l), and it is extracted with ethyl acetate (30 l). The combined organic extracts are concentrated in a vacuum, and the residue is filtered on silica gel with a mixture that consists of n-hexane/ethyl acetate. 2076 g (6819 mmol, 99.1%) of the title compound is isolated as a colorless oil.

¹H-NMR (300 MHz, CDCl₃) δ0.89+0.91+0.92+1.00 (6H), 1.41−1.88 (6H), 3.13+3.25 (1H), 3.34+3.37 (1H), 3.45 (1H), 3.86 (1H), 3.93+4.03 (1H), 4.44−4.69 (3H), 5.13−5.29 (2H), 5.67+5.89 (1H), 7.22−7.39 (5H) ppm.

Note: Chromatographic purification is also completely unnecessary at this point.

### Example 1b

(3S)-1-Benzyloxy-2,2-dimethyl-pentane-3-(tetrahydropyran-2(RS)-yloxy)-5-ol

BH₃-THF-complex (4200 ml, 1 M in THF) is added to a solution of (3S)-1-benzyloxy-2,2-dimethyl-3-(tetrahydropyran-2(RS)-yloxy)-pent-4-ene (2076 g, 6820 mmol) in THF (26 l) at 23° C. over a period of 20 minutes. After two hours, the solution is cooled to 3° C. and mixed with sodium hydroxide solution (3400 ml, 5% in water) over a period of 1 hour. It is again cooled to 0° C., and a solution of H₂O₂ (1690 ml, 30% in water) is added. After 1 hour at 4° C., the mixture is added in portions (10 l) to a sodium thiosulfate solution (about 5000 g in 17 l of water) and extracted with ethyl acetate (30 l). The combined organic extracts are concentrated in a vacuum, and the residue is filtered on silica gel with a mixture that consists of n-hexane/ethyl acetate. 1145 g of (3S)-1-benzyloxy-2,2-dimethyl-pentane-3-(tetrahydropyran-2(RS)-yloxy)-5-ol (3551 mmol, 52.1%) is isolated as a colorless oil, as well as 118 g of (3S)-1-benzyloxy-2,2-dimethyl-pentane-3,5-diol (495 mmol, 7.2%) and 172 g of (3S,4RS)-1-benzyloxy-2,2-dimethyl-pentane-3-(tetrahydropyran-2(RS)-yloxy)-4-ol (533 mmol, 7.8%).

¹H-NMR (300 MHz, CDCl₃) of (3S)-1-benzyloxy-2,2-dimethyl-pentane-3-(tetrahydropyran-2(RS)-yloxy)-5-ol δ0.88+0.93 (3H), 0.91+0.97 (3H), 1.39−1.91 (8H), 2.05 (1H), 3.08+3.22 (1H), 3.28+3.43 (1H), 3.44 (1H), 3.58−4.02 (4H), 4.44 (1H), 4.53 (1H), 4.67 (1H), 7.24−7.36 (5H) ppm.

¹H-NMR (300 MHz, CDCl₃) of (3S)-1-benzyloxy-2,2-dimethyl-pentane-3,5-diol δ0.89 (3H), 0.93 (3H), 1.64 (2H), 3.20 (1H), 3.31 (1H), 3.41 (1H), 3.72 (1H), 3.79-3.88 (3H), 4.51 (2H), 7.25-7.39 (5H) ppm.

### Example 1c

4(S)-[2-Methyl-1-benzyloxy-prop-2-yl]-2,2-dimethyl-[1,3]dioxane

Method 1

2,2-Dimethoxypropane (340 ml, 2775 mmol) and (±)-camphor-10-sulfonic acid (4.3 g, 18.5 mmol) are added to the solution of (3S)-1-benzyloxy-2,2-dimethyl-pentane-3,5-diol (118 g, 495 mmol) in CH₂Cl₂ (2.5 l), and the mixture is stirred for 16 hours at 23° C. The mixture is added in drops in saturated sodium bicarbonate solution and extracted with CH₂Cl₂. The organic extracts are washed with saturated sodium chloride solution, dried on sodium sulfate and concentrated after filtration in a vacuum. The residue is purified by chromatography with a mixture that consists of ethyl acetate/hexane, and 113 g of 4(S)-[2-methyl-1-benzyloxy-prop-2-yl]-2,2-dimethyl-[1,3]dioxane (406 mmol, 82.0%) is isolated as a colorless oil.

¹H-NMR (300 MHz, CDCl₃) δ0.88 (3H), 0.89 (3H), 1.29 (1H), 1.34 (3H), 1.41 (3H), 1.67 (1H), 3.14 (1H), 3.33 (1H), 3.80-3.89 (2H), 3.94 (1H), 4.48 (2H), 7.24-7.36 (5H) ppm.

Note: Chromatographic purification is also completely unnecessary at this point.

Method 2

A solution of (3S)-1-benzyloxy-2,2-dimethyl-pentane-3-(tetrahydropyran-2(RS)-yloxy)-5-ol (471 g, 1461 mmol) in acetone (2.3 l) is mixed with 2,2-dimethoxypropane (900 ml, 7345 mmol) and p-toluenesulfonic acid (27.8 g, 146 mmol), and the mixture is stirred for 22 hours at 23° C. The mixture is added in drops in saturated sodium bicarbonate solution, diluted with water (1 l) and extracted with CH₂Cl₂ (5 l). The organic extracts are washed with saturated sodium chloride solution, dried on sodium sulfate and concentrated after filtration in a vacuum. The residue is purified by chromatography with a mixture that consists of ethyl acetate/hexane, and 349 g of 4(S)-[2-methyl-1-benzyloxy-prop-2-yl]-2,2-dimethyl-[1,3]dioxane (1254 mmol, 85.8%) is isolated as a colorless oil, as well as 56 g of 2(RS),4(S)-[2-methyl-1-benzyloxy-prop-2-yl]-2-(1-hydroxybut-4-yl)-[1,3]dioxane (201 mmol, 13.8%).

¹H-NMR (300 MHz, CDCl₃) δ0.88 (3H), 0.89 (3H), 1.29 (1H), 1.34 (3H), 1.41 (3H), 1.67 (1H), 3.14 (1H), 3.33 (1H), 3.80-3.89 (2H), 3.94 (1H), 4.48 (2H), 7.24-7.36 (5H) ppm.

Note: Chromatographic purification is also completely unnecessary at this point.

### Example 1d

(4S)-4-(2-Methyl-1-hydroxy-prop-2-yl)-2,2-dimethyl-[1,3]dioxane

The solution of 4(S)-[2-methyl-1-benzyloxy-prop-2-yl]-2,2-dimethyl-[1,3]dioxane (31.9 g, 124 mmol) in ethanol (70 ml) is mixed with Pd/C (450 mg, 10%) and hydrogenated under an atmosphere of hydrogen at 23° C. until there is no more uptake. After filtration and removal of the solvent, 21.8 g of (4S)-4-(2-methyl-1-hydroxy-prop-2-yl)-2,2-dimethyl-[1,3]dioxane (116 mmol, 93.3%) is isolated as a colorless oil, which can be further reacted without purification.

¹H-NMR (300 MHz, CDCl₃) δ3.96 (1H), 3.87 (1H), 3.80 (1H), 3.55 (1H), 3.37 (1H), 2.99 (1H), 1.77 (1H), 1.45 (3H), 1.38 (3H), 1.36 (1H), 0.90 (3H), 0.88 (3H) ppm.

Note: Chromatographic purification is also completely unnecessary at this point.

### Example 1e

(4S)-4-(2-Methyl-1-oxo-prop-2-yl)-2,2-dimethyl-[1,3]dioxane

DMSO (21.1 ml, 297 mmol) and, after 10 minutes, the solution of (4S)-4-(2-methyl-1-hydroxy-prop-2-yl)-2,2-dimethyl-[1,3]dioxane (20.0 g, 106.2 mmol) in CH₂Cl₂ (0.5 l) are added at −70° C. to a solution that consists of oxalyl chloride (13.0 ml, 151.6 mmol) in CH₂Cl₂ (0.5 l). After 30 minutes, it is mixed with triethylamine (64.8 ml, 467 mmol) and stirred for 1 hour at −35° C. Water is added, and the mixture is extracted with CH₂Cl₂. The organic extracts are washed with saturated sodium chloride solution, dried on sodium sulfate and concentrated after filtration in a vacuum. 20.9 g of (4S)-4-(2-methyl-1-oxo-prop-2-yl)-2,2-dimethyl-[1,3]dioxane (maximum 106 mmol) is isolated as a pale yellow oil, which can be further reacted without purification.

¹H-NMR (300 MHz, CDCl₃) δ1.03 (3H), 1.08 (3H), 1.35 (3H), 1.39 (1H), 1.44 (3H), 1.70 (1H), 3.82-4.04 (3H), 9.59 (1H) ppm.

### Example 1f

(4S.3RS)-4-(2-Methyl-3-hydroxy-butan-2-yl)-2,2-dimethyl-[1,3]dioxane

A solution that consists of methylmagnesium bromide (120 ml, 3.0 M in diethyl ether) is cooled to 0° C. and mixed with the solution of (4S)-4-(2-methyl-1-oxo-prop-2-yl)-2,2-dimethyl-[1,3]dioxane (42.2 g, 227 mmol) in diethyl ether (800 ml) over a period of 2 hours. After 45 minutes, the mixture is poured into an ice-cold ammonium chloride solution and extracted with ethyl acetate. The organic extracts are washed with saturated sodium chloride solution, dried on sodium sulfate and concentrated after filtration in a vacuum. The residue is purified by chromatography with a mixture that consists of ethyl acetate/hexane, and 41.3 g of (4S,3RS)-4-(2-methyl-3-hydroxy-butan-2-yl)-2,2-dimethyl-[1,3]dioxane (204 mmol, 89.9%) is isolated as a colorless oil.

¹H-NMR (300 MHz, CDCl₃) δ0.70+0.87+0.91 (6H), 1.01−1.18 (3H), 1.31−1.49 (7H), 1.68−1.92 (1H), 3.68−4.01 (5H) ppm.

### Example 1g

(4S)-4-(2-Methyl-3-oxo-butan-2-yl)-2,2-dimethyl-[1,3]dioxane

Method 1

Molecular sieve (4 Å, 4.0 g), N-methylmorpholine-N-oxide (36 g, 307 mmol) and tetrapropylammonium-perruthenate (331 g, 8382 mmol) are added to a solution that consists of (4S,3RS)-4-(2-methyl-3-hydroxy-butan-2-yl)-2,2-dimethyl-[1,3]dioxane (41.3 g, 204 mmol) in CH₂Cl₂ (2.5 l). It is stirred overnight, filtered, and the residue is purified by filtration on silica gel with a mixture that consists of hexane/ethyl acetate. 38.6 g of (4S)-4-(2-methyl-3-oxo-butan-2-yl)-2,2-dimethyl-[1,3]dioxane (193 mmol, 94.5%) is isolated as a crystalline solid.

¹H-NMR (300 MHz, CDCl₃) δ1.06 (3H), 1.12 (3H), 1.34 (3H), 1.35 (1H), 1.43 (3H), 1.63 (1H), 2.16 (3H), 3.85 (1H), 3.96 (1H), 4.03 (1H) ppm.

Method 2

DMSO (1.9 ml, 26.8 mmol) and, after 10 minutes, the solution of (4S,3RS)-4-(2-methyl-3-hydroxy-butan-2-yl)-2,2-dimethyl-[1,3]dioxane (1.95 g, 9.64 mmol) in CH₂Cl₂ (36 ml) are added to a solution that consists of oxalyl chloride (1.49 ml, 13.45 mmol) in CH₂Cl₂ (36 ml) at −70° C. After 30 minutes, it is mixed with triethylamine (5.52 ml, 39.86 mmol) and stirred for 1 hour at −35° C. Water is added, and the mixture is extracted with CH₂Cl₂. The organic extracts are washed with saturated sodium chloride solution, dried on sodium sulfate and concentrated after filtration in a vacuum. It is purified by filtration on silica gel, and 1.36 g of (4S)-4-(2-methyl-3-oxo-butan-2-yl)-2,2-dimethyl-[1,3]dioxane (6.79 mmol, 70.5%) is isolated as a colorless solid.

Note: Purification can also take place at this point exclusively by recrystallization of the solid crude product.

### Example 1h

(4S)-4-(2-Methyl-3-oxo-hept-6-en-2-yl)-2,2-dimethyl-[1,3]dioxane

A solution of diisopropylamine (21.9 ml, 155.8 mmol) in THF (77 ml) is mixed under an atmosphere of argon at −30° C. with n-BuLi (57 ml, 143 mmol, 2.5 M in hexane), and it is stirred for 15 minutes at 0° C. It is mixed with toluene (77 ml), cooled to −70° C. and mixed with the solution that consists of (4S)-4-(2-methyl-3-oxo-butan-2-yl)-2,2-dimethyl-[1,3]dioxane (26.0 g, 129.9 mmol) in toluene (182 ml) and 1,3-dimethyltetrahydro-2(1H)-pyrimidinone (DMPU; 31.5 ml, 261 mmol). The temperature is allowed to increase within 1.5 hours to −20° C., the solution of allyl bromide (56 ml, 647 mmol) in toluene (130 ml) is added in drops within one hour and allowed to heat to 23° C. within 1.5 hours. While being cooled with ice, it is poured into a saturated ammonium chloride solution, diluted with water and extracted several times with ethyl acetate. The combined organic extracts are washed with saturated sodium chloride solution, dried on sodium sulfate, and the residue that is obtained after filtration and removal of the solvent is purified by chromatography on fine silica gel with a mixture that consists of n-hexane and ethyl acetate.

24.7 g of (4S)-4-(2-methyl-3-oxo-hept-6-en-2-yl)-2,2-dimethyl-[1,3]dioxane (102.8 mmol, 79%) is isolated as a colorless oil.

¹H-NMR (300 MHz, CDCl₃) δ5.81 (1H), 5.02 (1H), 4.95 (1H), 4.04 (1H), 3.95 (1H), 3.85 (1H), 2.60 (2H), 2.29 (2H), 1.62 (1H), 1.41 (3H), 1.32 (3H), 1.31 (1H), 1.13 (3H), 1.06 (3H) ppm.

### Example 2

In the example of the production of (4S)-4-(2-methyl-3-oxo-hept-6-en-2-yl)-2,2-dimethyl-[1,3]dioxane (Example 1; A-XIV, R^{1a}′═R^{1b}′═CH₃, R^{15a}═R^{15b}═CH₃, R^{2a}═H, R^{2b}═allyl) starting from pantolactone (A-II, R^{1a}′═R^{1b}′═CH₃), the process that is described in WO 99/07692 is compared to the new process that is described here with respect to the total yield. In this case, at each stage, the individual yields were averaged from several batches that were produced similarly to compare optionally present individual fluctuations. The result is presented in the following table:

| | Process | Process |
|---|---|---|
| | Analogous to | Described |
| | WO 99/07692 | Here |
| Total Yield (A-II to A-XIV): | 13.1 % v.E. | 37.2 % v.E. |
| Total Yield (A-Il to A-XIV): | 7.1 % of | 19.1 % of |
| | theory | theory |
| Average Yield per Stage | 78.6 % of | 87.5 % of |
| | theory | theory |

In this example, the total yield according to the new process is 269% of the process that is described in WO 99/07692.

The entire disclosure of all applications, patents and publications, cited herein and of corresponding German Application No. 101 64 592.9, filed Dec. 21, 2001, is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. C₁-C₆-Epothilone fragments of general formula I, 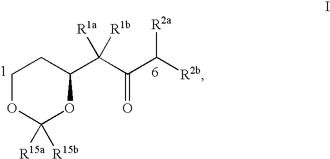
in which
R^{1a}, R^{1b} are the same or different and mean hydrogen, C₁-C₁₀-alkyl, aryl, C₇-C₂₀-aralkyl, or together mean a —(CH₂)ₘ group with m=2, 3, 4 or 5,
R^{2a}, R^{2b} are the same or different and mean hydrogen, C₁-C₁₀-alkyl, C₂-C₁₀-alkenyl, C₂-C₁₀-alkinyl, aryl, C₇-C₂₀-aralkyl or together mean a —(CH₂)ₙ group with n=2, 3, 4 or 5,
R^{15a}, R^{15b} are the same or different and mean hydrogen, C₁-C₁₀-alkyl, aryl, C₇-C₂₀-aralkyl, or together a —(CH₂)_{q} group,
q means 3 to 6, including all stereoisomers as well as mixtures thereof.

2. C₁-C₆-Epothilone fragments according to claim 1, in which
R^{1a}, R^{1b} are the same and mean C₁-C₆-alkyl, aryl, or together mean a —(CH₂)ₘ group with m=2, 3 or 4,
R^{2a}, R^{2b} are different and mean hydrogen, C₁-C₆-alkyl, C₂-C₁₀-alkenyl, C₂-C₁₀-alkinyl or C₇-C₂₀-aralkyl,
R^{15a}, R^{15b} are the same or different and mean hydrogen, C₁-C₅-alkyl, aryl, or C₇-C₂₀-aralkyl, or together mean a —(CH₂)_{q} group,
q means 3 to 6.

3. C₁-C₆-Epothilone fragments according to claim 1, in which
R^{1a}, R^{1b} are the same and mean methyl, ethyl, or aryl, or together a —(CH₂)ₘ group with=2 or 3,
R^{2a} means hydrogen,
R^{2b} means C₁-C₅-alkyl, C₂-C₆-alkenyl or C₂-C₆-alkinyl,
R^{15a}, R^{15b} are the same and mean C₁-C₃-alkyl, or together mean a —(CH₂)_{q} group, or
R^{15a} means hydrogen, and
R^{15b} means aryl,
q means 4 or 5.

4. (4S)-4-(2-Methyl-3-oxo-pent-2-yl)-2,2-dimethyl-[1,3]dioxane
(4S)-4-(2-Methyl-3-oxo-hex-2-yl)-2,2-dimethyl-[1,3]dioxane
(4S)-4-(2-Methyl-3-oxo-hept-2-yl)-2,2-dimethyl-[1,3]dioxane
(4S)-4-(2-Methyl-3-oxo-hept-2-yl)-2,2-dimethyl-[1,3]dioxane
(4S)-4-(2-Methyl-3-oxo-hex-5-en-2-yl)-2,2-dimethyl-[1,3]dioxane
(4S)-4-(2-Methyl-3-oxo-hept-6-en-2-yl)-2,2-dimethyl-[1,3]dioxane
(4S)-4-(2-Methyl-3-oxo-oct-6-en-2-yl)-2,2-dimethyl-[1,3]dioxane
(4S)-4-(2-Methyl-3-oxo-oct-7-en-2-yl)-2,2-dimethyl-[1,3]dioxane
(4S)-4-(2-Methyl-3-oxo-hex-5-in-2-yl)-2,2-dimethyl-[1,3]dioxane
(4S)-4-(2-Methyl-3-oxo-hept-6-in-2-yl)-2,2-dimethyl-[1,3]dioxane
(4S)-4-(2-Methyl-3-oxo-oct-6-in-2-yl)-2,2-dimethyl-[1,3]dioxane
(4S)-4-(2-Methyl-3-oxo-oct-7-in-2-yl)-2,2-dimethyl-[1,3]dioxane
(4S)-4-(2-Methyl-3-oxo-pent-2-yl)-2,2-(1,4-tetramethylene)-[1,3]dioxane
(4S)-4-(2-Methyl-3-oxo-hex-2-yl)-2,2-(1,4-tetramethylene)-[1,3]dioxane
(4S)-4-(2-Methyl-3-oxo-hept-2-yl)-2,2-(1,4-tetramethylene)-[1,3]dioxane
(4S)-4-(2-Methyl-3-oxo-hept-2-yl)-2,2-(1,4-tetramethylene)-[1,3]dioxane
(4S)-4-(2-Methyl-3-oxo-hex-5-en-2-yl)-2,2-(1,4-tetramethylene)-[1,3]dioxane
(4S)-4-(2-Methyl-3-oxo-hept-6-en-2-yl)-2,2-(1,4-tetramethylene)-[1,3]dioxane
(4S)-4-(2-Methyl-3-oxo-oct-6-en-2-yl)-2,2-(1,4-tetramethylene)-[1,3]dioxane
(4S)-4-(2-Methyl-3-oxo-oct-7-en-2-yl)-2,2-(1,4-tetramethylene)-[1,3]dioxane
(4S)-4-(2-Methyl-3-oxo-hex-5-in-2-yl)-2,2-(1,4-tetramethylene)-[1,3]dioxane
(4S)-4-(2-Methyl-3-oxo-hept-6-in-2-yl)-2,2-(1,4-tetramethylene)-[1,3]dioxane
(4S)-4-(2-Methyl-3-oxo-oct-6-in-2-yl)-2,2-(1,4-tetramethylene)-[1,3]dioxane
(4S)-4-(2-Methyl-3-oxo-oct-7-in-2-yl)-2,2-(1,4-tetramethylene)-[1,3]dioxane
(4S)-4-(2-Methyl-3-oxo-pent-2-yl)-2,2-(1,5-pentamethylene)-[1,3]dioxane
(4S)-4-(2-Methyl-3-oxo-hex-2-yl)-2,2-(1,5-pentamethylene)-[1,3]dioxane
(4S)-4-(2-Methyl-3-oxo-hept-2-yl)-2,2-(1,5-pentamethylene)-[1,3]dioxane
(4S)-4-(2-Methyl-3-oxo-hept-2-yl)-2,2-(1,5-pentamethylene)-[1,3]dioxane
(4S)-4-(2-Methyl-3-oxo-hex-5-en-2-yl)-2,2-(1,5-pentamethylene)-[1,3]dioxane
(4S)-4-(2-Methyl-3-oxo-hept-6-en-2-yl)-2,2-(1,5-pentamethylene)-[1,3]dioxane
(4S)-4-(2-Methyl-3-oxo-oct-6-en-2-yl)-2,2-(1,5-pentamethylene)-[1,3]dioxane
(4S)-4-(2-Methyl-3-oxo-oct-7-en-2-yl)-2,2-(1,5-pentamethylene)-[1,3]dioxane
(4S)-4-(2-Methyl-3-oxo-hex-5-in-2-yl)-2,2-(1,5-pentamethylene)-[1,3]dioxane
(4S)-4-(2-Methyl-3-oxo-hept-6-in-2-yl)-2,2-(1,5-pentamethylene)-[1,3]dioxane
(4S)-4-(2-Methyl-3-oxo-oct-6-in-2-yl)-2,2-(1,5-pentamethylene)-[1,3]dioxane
(4S)-4-(2-Methyl-3-oxo-oct-7-in-2-yl)-2,2-(1,5-pentamethylene)-[1,3]dioxane
(4S,2RS)-4-(2-Methyl-3-oxo-pent-2-yl)-2-phenyl-[1,3]dioxane
(4S,2RS)-4-(2-Methyl-3-oxo-hex-2-yl)-2-phenyl-[1,3]dioxane
(4S,2RS)-4-(2-Methyl-3-oxo-hept-2-yl)-2-phenyl-[1,3]dioxane
(4S,2RS)-4-(2-Methyl-3-oxo-hept-2-yl)-2-phenyl-[1,3]dioxane
(4S,2RS)-4-(2-Methyl-3-oxo-hex-5-en-2-yl)-2-phenyl-[1,3]dioxane
(4S,2RS)-4-(2-Methyl-3-oxo-hept-6-en-2-yl)-2-phenyl-[1,3]dioxane
(4S,2RS)-4-(2-Methyl-3-oxo-oct-6-en-2-yl)-2-phenyl-[1,3]dioxane
(4S,2RS)-4-(2-Methyl-3-oxo-oct-7-en-2-yl)-2-phenyl-[1,3]dioxane
(4S,2RS)-4-(2-Methyl-3-oxo-hex-5-in-2-yl)-2-phenyl-[1,3]dioxane
(4S,2RS)-4-(2-Methyl-3-oxo-hept-6-in-2-yl)-2-phenyl-[1,3]dioxane
(4S,2RS)-4-(2-Methyl-3-oxo-oct-6-in-2-yl)-2-phenyl-[1,3]dioxane
(4S,2RS)-4-(2-Methyl-3-oxo-oct-7-in-2-yl)-2-phenyl-[1,3]dioxane
(4S,2RS)-4-(2-Methyl-3-oxo-pent-2-yl)-2-(4-methoxy-phenyl)-[1,3]dioxane
(4S,2RS)-4-(2-Methyl-3-oxo-hex-2-yl)-2-(4-methoxy-phenyl)-[1,3]dioxane
(4S,2RS)-4-(2-Methyl-3-oxo-hept-2-yl)-2-(4-methoxy-phenyl)-[1,3]dioxane
(4S,2RS)-4-(2-Methyl-3-oxo-hept-2-yl)-2-(4-methoxy-phenyl)-[1,3]dioxane
(4S,2RS)-4-(2-Methyl-3-oxo-hex-5-en-2-yl)-2-(4-methoxy-phenyl)-[1,3]dioxane
(4S,2RS)-4-(2-Methyl-3-oxo-hept-6-en-2-yl)-2-(4-methoxy-phenyl)-[1,3]dioxane
(4S,2RS)-4-(2-Methyl-3-oxo-oct-6-en-2-yl)-2-(4-methoxy-phenyl)-[1,3]dioxane
(4S,2RS)-4-(2-Methyl-3-oxo-oct-7-en-2-yl)-2-(4-methoxy-phenyl)-[1,3]dioxane
(4S,2RS)-4-(2-Methyl-3-oxo-hex-5-in-2-yl)-2-(4-methoxy-phenyl)-[1,3]dioxane
(4S,2RS)-4-(2-Methyl-3-oxo-hept-6-in-2-yl)-2-(4-methoxy-phenyl)-[1,3]dioxane
(4S,2RS)-4-(2-Methyl-3-oxo-oct-6-in-2-yl)-2-(4-methoxy-phenyl)-[1,3]dioxane
(4S,2RS)-4-(2-Methyl-3-oxo-oct-7-in-2-yl)-2-(4-methoxy-phenyl)-[1,3]dioxane
(4S,2RS)-4-(2-Methyl-3-oxo-pent-2-yl)-2-(2-cyano-phenyl)-[1,3]dioxane
(4S,2RS)-4-(2-Methyl-3-oxo-hex-2-yl)-2-(2-cyano-phenyl)-[1,3]dioxane
(4S,2RS)-4-(2-Methyl-3-oxo-hept-2-yl)-2-(2-cyano-phenyl)-[1,3]dioxane
(4S,2RS)-4-(2-Methyl-3-oxo-hept-2-yl)-2-(2-cyano-phenyl)-[1,3]dioxane
(4S,2RS)-4-(2-Methyl-3-oxo-hex-5-en-2-yl)-2-(2-cyano-phenyl)-[1,3]dioxane
(4S,2RS)-4-(2-Methyl-3-oxo-hept-6-en-2-yl)-2-(2-cyano-phenyl)-[1,3]dioxane
(4S,2RS)-4-(2-Methyl-3-oxo-oct-6-en-2-yl)-2-(2-cyano-phenyl)-[1,3]dioxane
(4S,2RS)-4-(2-Methyl-3-oxo-oct-7-en-2-yl)-2-(2-cyano-phenyl)-[1,3]dioxane
(4S,2RS)-4-(2-Methyl-3-oxo-hex-5-in-2-yl)-2-(2-cyano-phenyl)-[1,3]dioxane
(4S,2RS)-4-(2-Methyl-3-oxo-hept-6-in-2-yl)-2-(2-cyano-phenyl)-[1,3]dioxane
(4S,2RS)-4-(2-Methyl-3-oxo-oct-6-in-2-yl)-2-(2-cyano-phenyl)-[1,3]dioxane
(4S,2RS)-4-(2-Methyl-3-oxo-oct-7-in-2-yl)-2-(2-cyano-phenyl)-[1,3]dioxane

5. Process for the production of compounds of general formula I according to claim 1, containing the synthesis steps (Diagram 1) of:
The conversion of a compound of general formula A-V 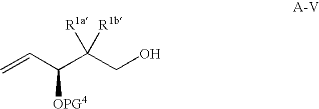
in which
R^{1a′} and R^{1b′} have the same meaning as R^{1a} and R^{1b} in claim 1, and OPG⁴ is a protective group that can be cleaved under acidic reaction conditions, preferably a tetrahydropyranyl group, into a compound of general formula A-VI 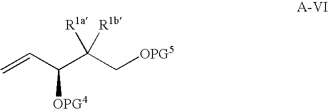
in which
OPG⁵ is a protective group that can be cleaved hydrogenolytically with use of a catalyst, preferably a benzyl group,
The conversion of the compound of general formula A-VI into a compound of general formula A-VII 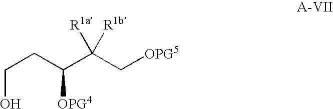
The conversion of the compound of general formula A-VII into a compound of general formula A-VIII 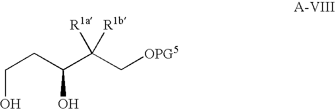
The conversion of the compound of general formula A-VIII into a compound of general formula A-IX 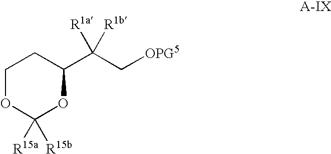
in which
R^{15a} and R^{15b} have the meanings that are indicated in claim 1, or optionally the direct conversion of the compound of general formula A-VII into the compound of general formula A-IX,
The conversion of the compound of general formula A-IX into a compound of general formula A-X 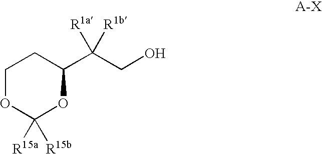
The conversion of the compound of general formula A-X into a compound of general formula A-XI 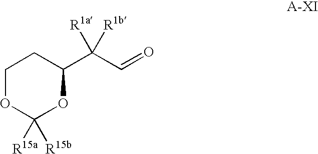
The conversion of the compound of general formula A-XI into a compound of general formula A-XII 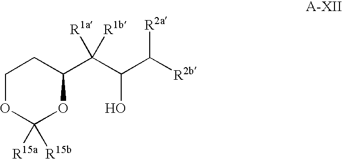
in which
R^{2a}′ and R^{2b}′ have the same meaning as R^{2a} and R^{2b} in claim 1, and the conversion of the compound of general formula A-XII into a compound of general formula A-XIII (=compound of general formula I) 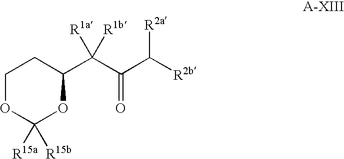
and optionally, if R^{2a}′ and/or R^{2b}′ in A-XIII is equal to hydrogen, the introduction of an additional radical R^{2a′}, which can have the meanings that are indicated in Formula I for R^{2a}, excluding hydrogen.
